(19) United States
(12) Patent Application Publication
(10) Pub. No.: US 2026/0165790 A1
(43) Pub. Date: Jun. 18, 2026

(54) MEDICAL IMAGE PROCESSING APPARATUS AND METHOD

(71) Applicant: CANON MEDICAL SYSTEMS CORPORATION, Otawara-shi (JP)

(72) Inventors: Ewan HEMINGWAY, Edinburgh (GB); John HIPWELL, Edinburgh (GB); Marcel SHEENY DE MORAES, Edinburgh (GB)

(73) Assignee: CANON MEDICAL SYSTEMS CORPORATION, Otawara-shi (JP)

(21) Appl. No.: 18/978,429

(22) Filed: Dec. 12, 2024

(51) Int. Cl. A61B 34/20; G06T 7/11; G06T 7/32; G16H 30/40

(52) U.S. Cl. CPC A61B 34/20; G06T 7/11; G06T 7/32; G16H 30/40; A61B 2034/2065

(57) ABSTRACT

A medical image processing apparatus comprising processing circuitry configured to: generate a first map representing a likely location of a medical device in an anatomical region of a patient or other subject, the first map being generated based on a two-dimensional (2D) image that has been acquired by a medical imaging apparatus, the 2D image representing the medical device in the anatomical region, generate a second map representing an expected location of the medical device in the anatomical region, the second map being generated based on volumetric imaging data representing the anatomical region and a plurality of registration parameters, wherein the plurality of registration parameter determines a pose of the medical imaging apparatus, compare the first and second maps with each other and update the plurality of registration parameters based on the comparison between the first and second maps.

### FIELD

The present invention relates to a medical image processing apparatus and a medical image processing method.

### BACKGROUND

2D/3D registration is a technique to estimate a spatial relationship between three-dimensional (3D) structures, e.g. volumetric imaging data, models of medical instruments or other 3D structures, and two-dimensional (2D) images thereof. For example, 2D/3D registration may be used in medical procedures to register volumetric imaging data representing an anatomical region or structure, which may be acquired by computed tomography (CT) and other X-ray scanners, such as nuclear magnetic resonance scanners, ultrasound scanners or other medical scanners, with a 2D image representing the anatomical region or structure. 2D/3D registration can be used to find an optimal geometric transformation that aligns a representation of the 3D structures with the 2D image. This may also be referred to as an optimisation problem.

An exemplary medical procedure, which requires 2D/3D registration, is transcatheter aortic valve implantation. To register the volumetric imaging data acquired by a CT scanner with the 2D image, a simulated X-ray image is generated based on the volumetric imaging data. This simulated X-ray image is also referred to as a digitally reconstructed radiograph (DRR). A pose, e.g. a position and angle, of an X-ray source of a medical imaging apparatus, which is used to acquire the 2D image, can be estimated by comparing the 2D image with the DRR. An optimization process can be used to determine the degrees of freedom, which may include six degrees of freedom, to determine the pose of the X-ray source.

The 2D image representing the anatomical region or structure may be part of a live image sequence acquired by a medical imaging method, such as fluoroscopy or another medical imaging method. The 2D image may also be referred to an intraoperative 2D image. When the field of view of the 2D image is reduced, which may be common during the medical procedure, 2D/3D registration may be difficult and/or ambiguous. The simulated X-ray image and the 2D image may be manually aligned, which may be very time consuming.

### DESCRIPTION

Embodiments are now described by way of non-limiting example with reference to the accompanying drawings in which:

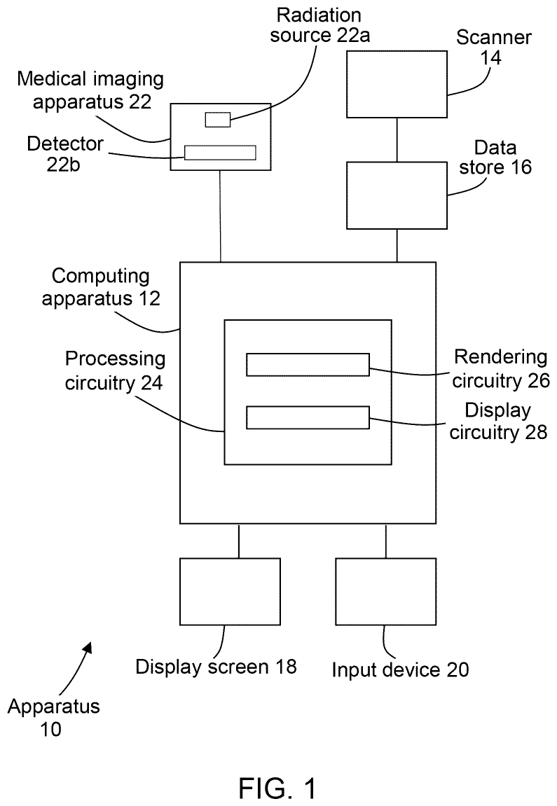

FIG. 1 is a schematic illustration of a medical image processing apparatus according to an embodiment;

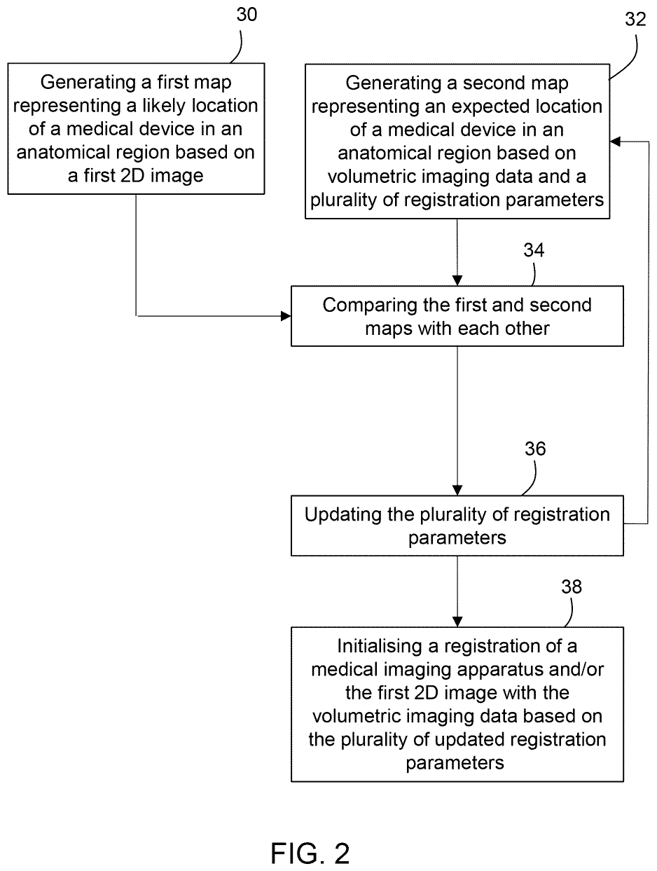

FIG. 2 is a flow chart illustrating in overview a process of an embodiment;

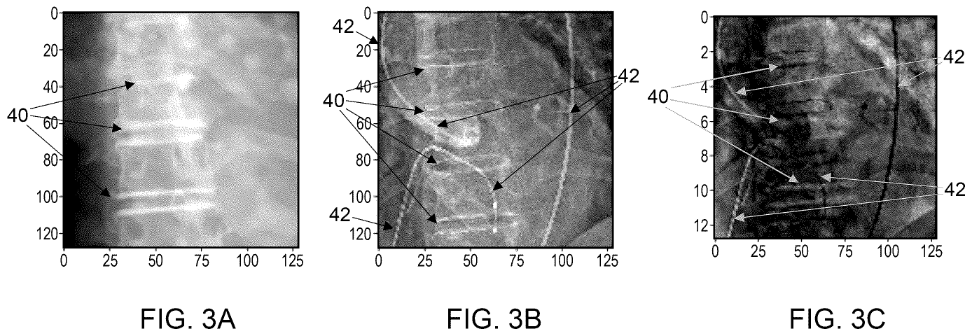

FIG. 3A illustrates a 2D image representing an anatomical region of a patient or other subject, the 2D image being a digitally reconstructed radiograph that has been generated from volumetric image data representing the anatomical region;

FIG. 3B illustrates a 2D image representing the anatomical region of FIG. 3A, the 2D image having been acquired using fluoroscopy;

FIG. 3C illustrates a 2D image representing a difference between the 2D image of FIG. 3A and the 2D image of FIG. 3B;

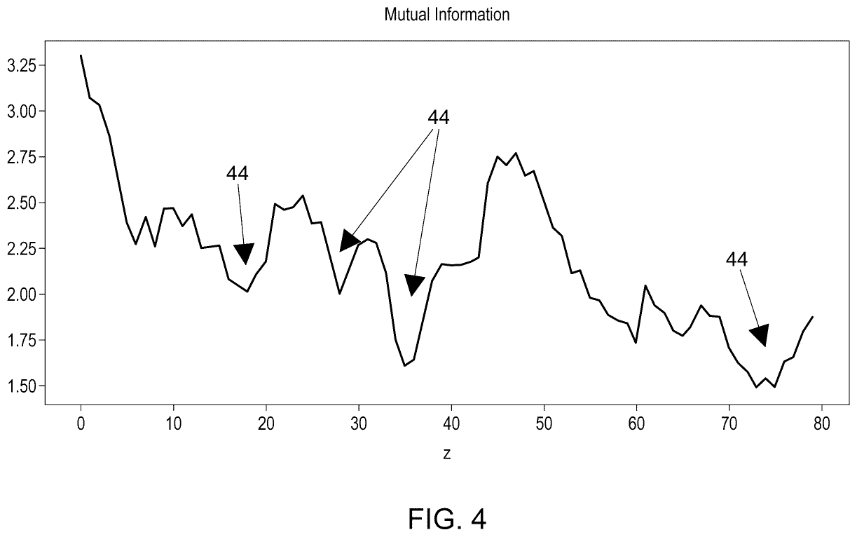

FIG. 4 illustrates a graph representing mutual information between the 2D image of FIG. 3A and the 2D image of FIG. 3B;

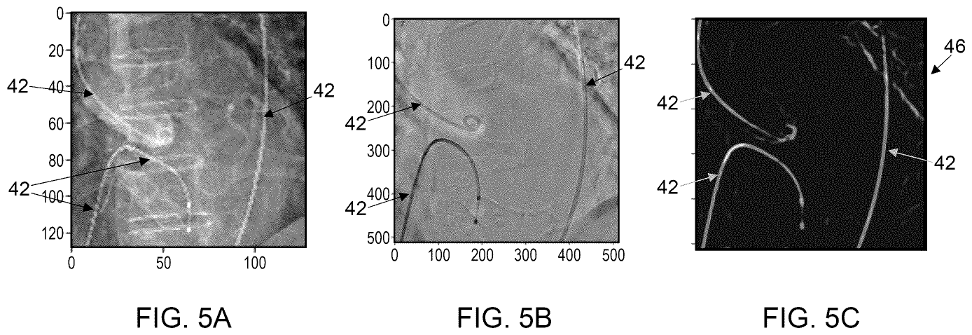

FIG. 5A illustrates the 2D image of FIG. 3B;

FIG. 5B illustrates the 2D image of FIG. 5A with a background removed;

FIG. 5C illustrates an exemplary first map, which has been generated based on the 2D image of FIG. 5B;

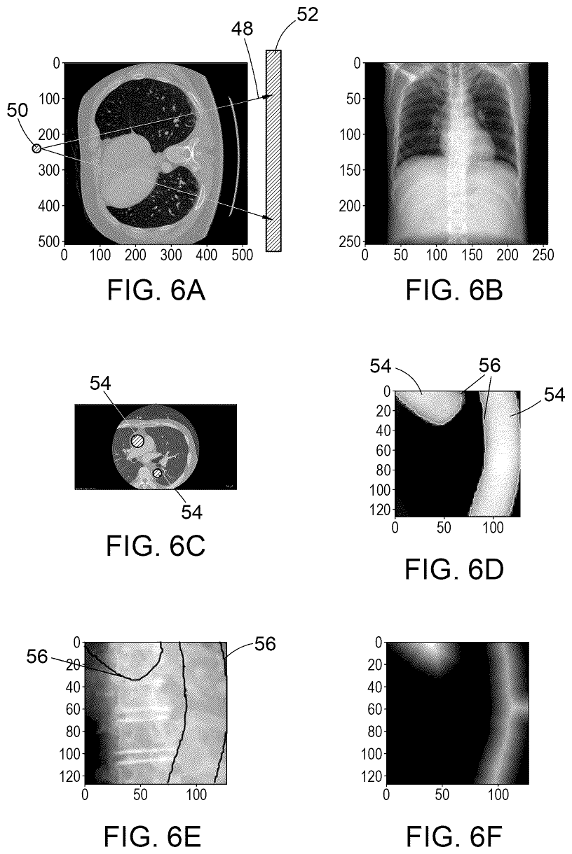

FIG. 6A illustrates a 2D image representing a sectional view of volumetric imaging data representing the anatomical region;

FIG. 6B illustrates a 2D image that has been generated by rendering the volumetric imaging data represented by the 2D image of FIG. 6A;

FIG. 6C illustrates an image representing a sectional view of volumetric imaging data representing the anatomical region with a segmentation of an aorta;

FIG. 6D illustrates a 2D image representing a segmented anatomical region, the segmented anatomical region having been projected without modification;

FIG. 6E illustrates the 2D image of FIG. 3A including an outline of the aorta;

FIG. 6F illustrates an exemplary distance map that has been generated based on 2D image illustrated in FIG. 6D;

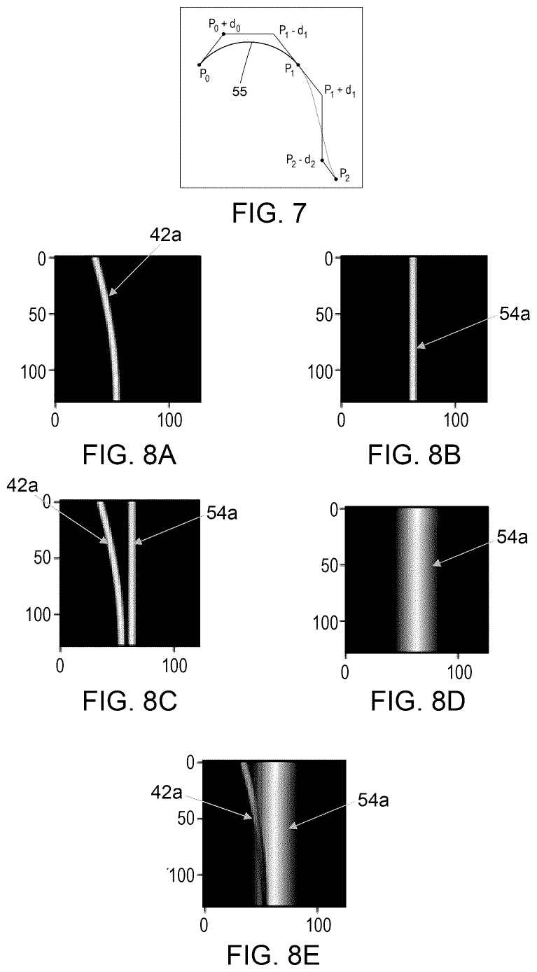

FIG. 7 illustrates an exemplary spline representing an expected location and/or route of a medical device in the volumetric imaging data representing the anatomical region;

FIG. 8A illustrates a 2D image representing a simulated medical device;

FIG. 8B illustrates a 2D image representing a simulated anatomical region;

FIG. 8C illustrates a 2D image representing a difference between the 2D image of FIG. 8A and the 2D image of FIG. 8B;

FIG. 8D illustrates a 2D image representing a simulated anatomical region with an increased size or dimension of the simulated anatomical region;

FIG. 8E illustrates a 2D image representing a difference between the 2D image of FIG. 8A and the 2D image of FIG. 8D;

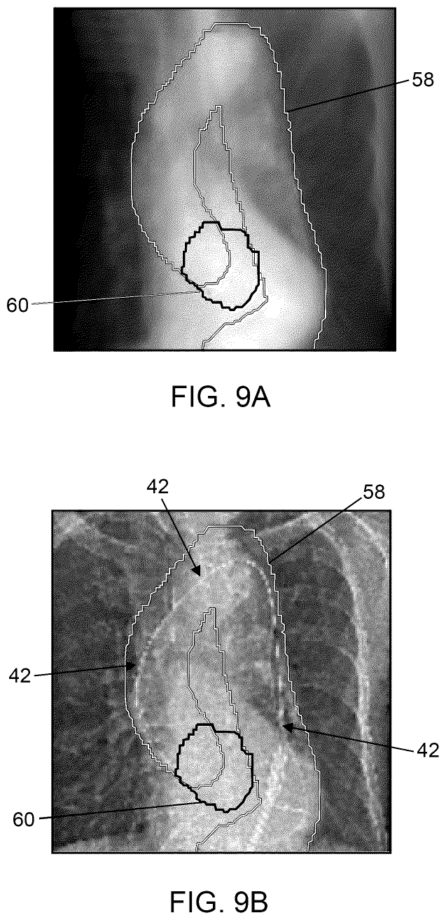

FIG. 9A illustrates a 2D image representing an anatomical region of a patient or other subject, the 2D image being a digitally reconstructed radiograph on which planning data has been projected; and

FIG. 9B illustrates a 2D image representing the anatomical region, the 2D image having been acquired using fluoroscopy and planning data having been projected thereon.

Certain embodiments provide a medical image processing apparatus comprising processing circuitry configured to generate a first map representing a likely location of a medical device in an anatomical region of a patient or other subject, the first map being generated based on a two-dimensional (2D) image that has been acquired by a medical imaging apparatus, the 2D image representing the medical device in the anatomical region, generate a second map representing an expected location of the medical device in the anatomical region, the second map being generated based on volumetric imaging data representing the anatomical region and a plurality of registration parameters, wherein the plurality of registration parameters determines a pose of the medical imaging apparatus, compare the first and second maps with each other and update the plurality of registration parameters based on the comparison between the first and second maps.

The processing circuitry may be configured to initialise a registration of at least one of: the medical imaging apparatus and/or a 2D image sequence with the volumetric imaging data, e.g. based on a plurality of updated registration parameters. The 2D image sequence may comprise at least the 2D image and/or one or more subsequent 2D images representing the medical device in the anatomical region.

The expected location of the medical device in the anatomical region may be part of planning data. The processing circuitry may be configured to generate the second map based on the planning data.

The processing circuitry may be configured to project the planning data onto one or more 2D images, e.g. based on a plurality of updated registration parameters. The one or more 2D images may comprise at least the 2D image and/or the one or more subsequent 2D images representing the medical device in the anatomical region.

The processing circuitry may be configured to determine the planning data based on a medical procedure to be carried out.

The planning data may be defined by a user.

The planning data may comprise at least one of: a route of the medical device in the anatomical region and/or a location of an anatomical structure.

The medical procedure to be carried out may comprise a transcatheter aortic valve implantation procedure. The anatomical region may comprise an aorta of a patient or other subject.

The processing circuitry may be configured to perform one or more iterations of at least one of: generating the first map, generating the second map, comparing the first and second maps with each other and/or updating the plurality of registration parameters based on the comparison between the first and second maps.

In a first iteration, the plurality of registration parameters may comprise a plurality of initial registration parameters. In each subsequent iteration, the plurality of registration parameters may comprise a plurality of updated registration parameters. The plurality of updated registration parameters may comprise at least one registration parameter that has been updated relative to a corresponding previous registration parameter. In each subsequent iteration, the processing circuitry may be configured to generate the second map based on a plurality of updated registration parameters.

The processing circuitry may be configured to detect the medical device in the 2D image. The processing circuitry may be configured to use a filtering method and/or a machine learning method to detect the medical device in the 2D image.

The processing circuitry may be configured to segment the anatomical region in the volumetric imaging data. The processing circuitry may be configured to project the segmented anatomical region onto an image plane, e.g. to generate an image representing the segmented anatomical region. The segmented anatomical region may be projected onto the image plane based on the plurality of registration parameters.

The processing circuitry may be configured to generate a binary image representing the segmented anatomical region, e.g. based on the generated image. The processing circuitry may be configured to increase a size or dimension of the segmented anatomical region. The processing circuitry may be configured to generate a distance map based on the generated image.

The processing circuitry may be configured to generate the second map based on one or more properties of the medical device.

The 2D image may comprise a fluoroscopic image. A range of a field of view of the 2D image may be reduced to focus on or zoom in the anatomical region.

The anatomical region may comprise an artery, vein or an organ of the patient or other subject. The organ may comprise a cylindrical or tubular shape.

Certain embodiments provide a medical image processing method comprising generating a first map representing a likely location of a medical device in an anatomical region of a patient or other subject, the first map being generated based on a two-dimensional (2D) image that has been acquired by a medical imaging apparatus, the 2D image representing the medical device in the anatomical region, generating a second map representing an expected location of the medical device in the anatomical region, the second map being generated based on volumetric imaging data representing the anatomical region and a plurality of registration parameters, wherein the plurality of registration parameters determines a pose of the medical imaging apparatus, comparing the first and second maps with each other and updating the plurality of registration parameters based on the comparison between the first and second maps.

A medical image processing apparatus 10 according to an embodiment is schematically illustrated in FIG. 1. The medical image processing apparatus 10 comprises a computing apparatus 12, which may be provided in the form of a personal computer (PC) or workstation. In this embodiment, the computing apparatus 12 is connected to a scanner 14, e.g. via a data store 16. However, it will be appreciated that in other embodiments, the medical image processing apparatus may not be connected or coupled to any scanner.

The medical image processing apparatus 10 further comprises one or more display screens 18 and an input device or devices 20, such as a computer keyboard, mouse or trackball.

In the present embodiment, the scanner 14 is a computed tomography (CT) scanner. However, it will be appreciated that in other embodiments the scanner may comprise another medical scanner, such as a nuclear magnetic resonance scanner, an ultrasound scanner or another medical scanner. The scanner 14 is configured to generate volumetric image data representing an anatomical region of a patient or other subject.

Volumetric image data comprises a plurality of voxels arranged in a three-dimensional (3D) grid. Each voxel has a voxel value associated with it. The voxel values represent measurements of a physical parameter. For example, in the case of CT scans, the voxel values represent the opacity of those voxels to X-rays, i.e. their X-ray stopping power. X-ray stopping power is measured in Hounsfield units (HUs) which is closely correlated with density (mass per unit volume).

In the present embodiment, volumetric image data sets obtained by the scanner 14 are stored in the data store 16 and subsequently provided to the computing apparatus 12. In an alternative embodiment, volumetric image data sets may be supplied from a remote data store (not shown). The data store 16 or remote data store may comprise any suitable form of memory storage.

In the present embodiment, the computing apparatus 12 is connected to a medical imaging apparatus 22. The medical imaging apparatus 22 is configured to acquire a first two-dimensional (2D) image representing a medical device in the anatomical region. The medical device may also be referred to as a tool, instrument or interventional object.

In this embodiment, the first 2D image is part of an image sequence, such as a live image sequence, representing the anatomical region. The image sequence, e.g. the first 2D image, has been acquired by a fluoroscopy method or another medical imaging method. The first 2D image may also be referred to as a frame of the image sequence of the anatomical region. The medical imaging apparatus 22 is configured to perform a fluoroscopy method, e.g. to acquire the image sequence of the anatomical region. The image sequence may be part of a live video representing the medical device in the anatomical region. The medical imaging apparatus 22 comprises a radiation source 22a, such as an X-ray source, and a detector 22b, such as a fluorescent screen. For example, the medical imaging apparatus 22 is provided in the form of a fluoroscope. In use, a patient or other subject is placed between the X-ray source and the fluorescent screen. In use, X-rays emitted by the X-ray source pass through the patient or other subject. The X-rays are attenuated as they pass through the different tissues of the patient's or other subjects' body. X-rays that have passed through the patient's or other subjects' body are detected on the fluorescent screen. Images on the screen are produced as the X-rays that have passed through the patient's or other subjects' body interact with atoms in the screen through the photoelectric effect. The first 2D image may also be referred to as an intraoperative image. The image sequence may also be referred to as a 2D medical image sequence. In this embodiment, a pose, e.g. a position and orientation, of the radiation source 22a relative to the detector 22b is fixed. For example, the radiation source 22a and the detector 22b may be mounted on a support, such as an arm, C-arm or other support, in a fixed pose relative to each other. It will be appreciated that in other embodiments, the medical imaging apparatus may be differently configured or arranged. For example, in such other embodiments, the medical imaging apparatus may comprise another radiation source and/or another detector and/or at least one of the radiation source and detector may be moveable relative to at least one other of the radiation source and detector.

The computing apparatus 12 comprises a processing circuitry 24 for processing of data. The processing circuitry 24 comprises a central processing unit (CPU) and Graphical Processing Unit (GPU). The processing circuitry 24 provides a processing resource for automatically or semi-automatically processing volumetric image data sets and/or medical image data set. In other embodiments, the data to be processed may comprise any image data, which may not be medical image data.

In the present embodiment, the computing apparatus 12 comprises rendering circuitry 26 configured to generate a second 2D image from volumetric image data representing the anatomical region. For example, the processing circuitry 24 may comprise the rendering circuitry 26. In this embodiment, the second 2D image comprises a digitally reconstructed radiograph representing the anatomical region. The second 2D image may also be referred to as a planning image. However, it will be appreciated that in other embodiments, the second 2D image may comprise an image projected using another projection and/or simulation method.

In the present embodiment, the processing circuitry 24 comprises display circuitry 28 configured to display the first and/or second 2D images to a user on the display screen 18.

In the present embodiment, the circuitries 24, 26, 28 are each implemented in the CPU and/or GPU by means of a computer program having computer-readable instructions that are executable to perform one or more operations of the medical image processing apparatus 10 and/or a medical image processing method of an embodiment described herein. In other embodiments, the circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for the sake of clarity.

FIG. 2 is a flow chart illustrating in overview a process of an embodiment.

At stage 30, the processing circuitry 24 is configured to generate a first map representing a location of a medical device in an anatomical region of a patient or other subject. The location of the medical device in the anatomical region may also be referred to as a likely location in the anatomical region. The processing circuitry 24 is configured to generate the first map based on the first 2D image mentioned above. The first map may also be referred to as a tool likelihood map, a tool likelihood image or a first generated image. The medical device may comprise a catheter, probe, such as an ultrasound probe, stent, clip, replacement aortic valve and/or another medical device.

At stage 32, the processing circuitry 24 is configured to generate a second map representing an expected location of the medical device in the anatomical region. The second map may be understood as a simulation of the first 2D image, e.g. a location of the medical device in the anatomical region. The expected location of the medical device in the anatomical region is part of planning data, such as pre-operative planning data. The planning data may further comprise a location of an anatomical structure, a route, such as an access route, intervention route or other route, of the medical device in the anatomical region and/or other information of a medical procedure or medical intervention to be carried out. In some embodiments, the processing circuitry 24 is configured to determine the planning data based on the medical procedure to be carried out. For example, the processing circuitry 24 is configured to determine the planning data based on a standardised plan for the medical procedure. In other embodiments, the planning data is defined by a user. The medical procedure may also be referred to as an intervention. The second map may also be referred to as a tool probability map or image, probability map or image, 2D probability map or image or second generated image. The generation of the second map may facilitate the solving of the optimisation problem mentioned above. The planning data may also be referred to as planning information, such as pre-operative planning information.

The processing circuitry 24 is configured to generate the second map based on volumetric imaging data representing the anatomical region, as will be described below in more detail. For example, the rendering circuitry 26 is configured to generate the second map based on volumetric image data received from the scanner 14 or the data store 16.

In this embodiment, the volumetric imaging data comprises volumetric imaging data acquired by a CT scanner. The volumetric imaging data may also be referred to as a medical image volume (CT) or 3D CT volume. It will be appreciated that in other embodiments, the volumetric imaging data may be acquired by any other suitable scanner.

The processing circuitry 24 is further configured to generate the second map based on the plurality of registration parameters. In this embodiment, the registration parameters determine a pose of the medical imaging apparatus 22.

In this embodiment, the medical imaging apparatus 22 is moveable relative to the anatomical region of the patient or other subject. The medical imaging apparatus 22 comprises a plurality of degrees of freedom. For example, the medical imaging apparatus 22 comprises six degrees of freedom, such as three translational degrees of freedom and three rotational degrees of freedom. The medical imaging apparatus 22 is moveable in an x-direction, y-direction and z-direction, e.g. in a direction of an x-axis, y-axis and z-axis of a three-dimensional coordinate system, respectively. The medical imaging apparatus 22 is rotatable around the x-axis, y-axis and z-axis. The pose of the medical imaging apparatus 22 can be characterised in the three-dimensional coordinate system by six coordinate that define the position along the x-, y- and z-axes and a respective rotation about the x-, y- and z-axes. The rotation of the medical imaging apparatus 22 may also be referred to as an orientation or angle relative to at least one of the x-, y- and z-axes. The registration parameter may also be referred to as 2D/3D registration parameters. It will be appreciated that in some embodiments, the registration parameters may determine pose of a part of the medical imaging apparatus. For example, the part of the medical imaging apparatus 22 comprises the radiation source 22a, e.g. the X-ray source. In such embodiments, the features described above may apply to the radiation source 22a.

At stage 34, the processing circuitry 24 is configured to compare the first and second maps with each other. For example, the processing circuitry 24 is configured to compare the first and second maps with each other based on one or more image metrics, such as one or more image similarity metrics. For example, the image metrics comprise a negative normalized cross correlation, mutual information or other suitable image metrics. The processing circuitry 24 is configured to determine one or more similarities or an equivalency between the first and second maps. This may allow for disambiguation of the alignment between the anatomical region represented by the volumetric imaging data and the anatomical region represented by the first 2D image. As such, the comparison of the first and second maps may allow for disambiguation of the optimisation problem mentioned above and/or facilitate an optimisation of the registration parameters. For example, by comparing the likely location of the medical device in the anatomical region with the expected location of the medical device in the anatomical region, an estimation of the pose of the medical imaging apparatus 22 may be facilitated.

At stage 36, the processing circuitry 24 is configured to update or optimise the registration parameters based on the comparison between the first and second maps. The processing circuitry 34 may be configured to use an optimisation algorithm or process. The optimisation algorithm or process may comprise a machine learning algorithm or process, such as a gradient descent algorithm or any other suitable machine learning algorithm, to optimise or update the registration parameters, e.g. to minimise the image metrics.

The processing circuitry is configured to perform one or more iterations of stages 30 to 36.

In some embodiments, in each iteration, the processing circuitry 24 is configured to repeat stages 32 to 36 one or more times e.g. until the image metrics are minimised. For example, stages 32 to 36 may be repeated one or more times using the same first map.

In some embodiments, the processing circuitry 24 is configured to repeat stages 30 to 36. For example, in one or more subsequent iterations, stages 30 to 36 may be repeated for one or more subsequent 2D images of the image sequence. In each iteration, the processing circuitry 24 is configured to generate a first map based on a first 2D image of the image sequence and to perform stages 32 to 36 based on the first map. As described above, stages 32 to 36 may be repeated one or more times using the same first map.

In the above embodiments, in a first iteration, the plurality of registration parameters comprises a plurality of initial registration parameters at stage 32. The processing circuitry 24 may be configured to determine the initial registration parameters from a graph representing mutual information between the first 2D image and the second 2D image or another image representing volumetric imaging data. The plurality of initial registration parameters may also be referred to as crude registration parameters. In each subsequent iteration, the plurality of registration parameters comprises a plurality of updated registration parameters at stage 32. The plurality of updated registration parameters comprises at least one registration parameter that has been updated relative to a corresponding previous registration parameter. In each subsequent iteration, the processing circuitry 24 is configured to generate the second map based on the plurality of updated registration parameters. This may allow for an alignment of the second map with the medical device in the first 2D image to be updated and/or improved. The generation of the second map based on the updated registration parameters may also be referred to as updating the second map.

At stage 38, the processing circuitry 24 is configured to initialise a registration of the medical imaging apparatus 22 with the volumetric imaging data based on the plurality of updated registration parameters. This also initialises the registration of the first 2D image and/or one or more subsequent first 2D images of the image sequence, such as the image sequence, with the volumetric imaging data based on the plurality of updated registration parameters. The registration of the subsequent first 2D image of the 2D image sequence with the volumetric imaging data may be constrained to registration parameters that are the same or similar to the updated registration parameters. The processing circuitry 24 is configured to store the plurality of updated registration parameters as constraints for the registration of the medical imaging apparatus 22 and/or the first 2D images with the volumetric imaging data, e.g. in a memory storage of the computing apparatus 12.

FIG. 3A illustrates a 2D image representing an anatomical region of a patient or other subject. The anatomical region comprises the aorta of the heart of the patient or other subject. However, it will be appreciated that in other embodiments, the anatomical region may comprise another anatomical structure or region. For example, in other embodiments, the anatomical region may comprise another artery, a vein, an organ or another anatomical structure of the patient or other subject. In such other embodiments, the organ may comprise a cylindrical or tubular shape. For example, the organ may comprise the oesophagus, rectum or the like. The term anatomical structure may be understood as encompassing an organ or any other anatomical structure of the patient's or other subject's body. The anatomical structure may be part of the anatomical region.

The 2D image illustrated in FIG. 3A is a digitally reconstructed radiograph that has been generated from the volumetric image data representing the anatomical region. In FIG. 3A, it is difficult to see any features of the aorta due to the bones of the skeleton of the patient or other subject being dominant in the 2D image. The bones of the skeleton are indicated in FIG. 3A by reference numerals 40. The 2D image illustrated in FIG. 3A represents a planning image comprising the planning data for a medical procedure to be carried out. In this embodiment, the medical procedure to be carried out comprises a transcatheter aortic valve implantation (TAVI) procedure. In this embodiment, access for the medical device may be provided via the aorta. However, it will be appreciated that in other embodiments, the medical procedure to be carried out may comprise a different medical procedure. For example, in other embodiments, the medical procedure may comprise a vascular intervention procedure, ultrasound procedure, such as prostate/rectal ultrasound procedure or endoscopic ultrasound, or the like. The 2D image illustrated in FIG. 3A can be considered as the second 2D image described above.

FIG. 3B illustrates a 2D image representing the anatomical region shown in FIG. 3A. The 2D image shown in FIG. 3B has been acquired using the fluoroscopy method described above. In the 2D image illustrated in FIG. 3B, the bones 40 of the skeleton of the patient or other subject are visible. Parts of a medical device 42, which in this embodiment, is in the form of a catheter, are also visible in the 2D image illustrated in FIG. 3B. The 2D image illustrated in FIG. 3B can be considered as the first 2D image described above.

In FIGS. 3A and 3B, the field of view is reduced. It can be very difficult to perform 2D/3D registration when a field of view of the 2D images is reduced. However, it may be necessary to reduce the field of view in order to focus on or zoom in the anatomical region. In this embodiment, the field of view is reduced to focus on the aorta and not the skeleton of the patient or other subject.

FIG. 3C illustrates a 2D image representing a difference between the second 2D image illustrated in FIG. 3A and the first 2D image illustrated in FIG. 3B. The 2D image illustrated in FIG. 3C has been obtained by subtracting the first 2D image illustrated in FIG. 3B from the second 2D image illustrated in FIG. 3A. It can be seen in the 2D image illustrated in FIG. 3C that only the bones 40 of the skeleton and the medical device 42 are clearly visible. This may make a registration of the medical imaging apparatus 22 with the volumetric imaging data difficult and/or ambiguous.

FIG. 4 illustrates a graph representing mutual information between the 2D image illustrated in FIG. 3A and the 2D image illustrated in FIG. 3B. The graph shown in FIG. 4 may also be referred to as a similarity graph. The graph shown in FIG. 4 has been generated by moving a virtual x-ray detector, which will be described below in more detail, in at least one direction. In this embodiment, the virtual x-ray detector has been moved in the z-direction, which shown on the y-axis of the graph in FIG. 4. The z-direction represents a degree of freedom of the virtual x-ray detector. Each time the virtual x-ray detector is moved, a digitally reconstructed radiograph is generated from the volumetric image data representing the anatomical region. This radiograph is compared with a 2D image representing the anatomical region, which has been acquired using the fluoroscopy method mentioned above. An image metric is determined, based on the comparison. In this embodiment, the image metric comprises mutual information. It can be seen in FIG. 4 that there are a number of local minima 44, which are indicative of the bones 40 of the skeleton, illustrated in FIGS. 3A to 3C. The repetitive nature of the bones 40 of the skeleton in FIGS. 3A to 3C may make the alignment between the corresponding anatomic structures in FIGS. 3A and 3B ambiguous, which in turn may make the 2D/3D registration difficult. The 2D/3D registration may be difficult in this example, even though the virtual x-ray detector was only moved in a single direction, thereby changing only a single degree of freedom of the virtual x-ray detector.

2D/3D registration may be used to bring the planning data, e.g. represented by the 2D image illustrated in FIG. 3A, and intervention data, e.g. represented by the image illustrated in FIG. 3B into a same coordinate frame. The process illustrated in FIG. 2 may allow for an optimisation of the registration parameters that determine the pose of the medical imaging apparatus 22. This may allow for a more robust initialisation of the registration of the medical imaging apparatus 22 with the volumetric imaging data. This in turn may allow for a more robust registration of the volumetric imaging data with the image sequence.

FIGS. 5A to 5C illustrate the step of generating the first map, as described in relation to stage 30 illustrated in FIG. 2. The 2D image shown in FIG. 5A corresponds to the first 2D image illustrated in FIG. 3B. The 2D image shown in FIG. 5B illustrates the first 2D image shown in FIG. 5A with a background removed. The 2D image shown in FIG. 5C illustrates an exemplary first map, which has been generated based on the first 2D image shown in FIG. 5B.

In the present embodiment, the processing circuitry 24 is configured to detect the medical device 42 in the first 2D image representing the anatomical region. The processing circuitry 24 is configured to detect the location of the medical device 42 in the first 2D image representing the anatomical region using a filtering method. For example, the processing circuitry 24 may be configured to apply one or more filters to the first 2D image. In this embodiment, the filters were applied to the first 2D image illustrated in FIG. 5A. The filters may also be referred to as classical filters. The filters comprise a smoothing filter, such as a median filter, an anisotropic diffusion filter or any other suitable smoothing filter. The processing circuitry 24 is configured to apply the smoothing filter to remove a background of the first 2D image. In this embodiment, the smoothing filter has been applied to the first 2D image shown in FIG. 5A. The filtered first 2D image is shown FIG. 5B.

The filters may further comprise a vesselness filter, such as a Frangi vesselness filter or any other suitable vesselness filter. The processing circuitry 24 is configured to apply the vesselness filter to the first 2D image, e.g. to detect the medical device 42 in the first 2D image representing the anatomical region. The processing circuitry 24 is configured to extract the medical device 42 from the first 2D image to generate the first map. In this embodiment, the medical device 42 has been extracted from the first 2D image illustrated in FIG. 5A to generate the first map 46 illustrated in FIG. 5C. As can been seen in FIG. 5C, the first map represents the likely location of the medical device 42 in the anatomical region.

In other embodiments, the processing circuitry is configured to detect the medical device in the first 2D image and/or extract the medical device from the first 2D image using a machine learning model, such as a convolutional neural network (CNN).

FIGS. 6A to 6F illustrate the step of generating the second map, as described in relation to stage 32 illustrated in FIG. 2.

FIG. 6A illustrates a 2D image representing a sectional view of volumetric imaging data representing the anatomical region. FIG. 6B illustrates a 2D image that has been generated by rendering the volumetric imaging data represented by the 2D image in FIG. 6A. In this embodiment, FIG. 6B illustrates a digitally reconstructed radiograph (DRR) of the volumetric image data representing the anatomical region illustrated in FIG. 6A. A DRR can be understood as a simulated or synthetic image generated from source volumetric image data. The rendering circuitry 26 is configured to generate a DRR from the volumetric image data representing the anatomical region, e.g. by casting a plurality of rays 48 with a fixed initial energy from a virtual source 50 through the volumetric image data. In this embodiment, the volumetric image data comprises CT data, the plurality of rays comprises a plurality of X-rays 48 and the virtual source 50 comprises a virtual x-ray source. The plurality of X-rays 48 are attenuated as they move through the CT data and energy in the attenuated X-rays 48 is measured, when the X-rays 48 are incident on a detector 52. In this embodiment, the detector 52 is provided in the form of a virtual x-ray detector. The detector 52 represents an image plane. The energy absorbed by the CT data is determined and converted into a pixel value. For example, the rendering circuitry 26 can be configured to calculate a pixel value at a location where an X-ray is incident on the detector 52 as a weighted average of the values of the voxels through which the X-ray passes, e.g. using Siddon's method. It will be appreciated that in other embodiments, another rendering process may be used to generate a 2D image from the volumetric imaging data representing the anatomical region. The other rendering process may comprise a more complex physics-based process, such as a DeepDRR process, as described by Unberath M. et al in “DeepDRR—A Catalyst for Machine Learning in Fluoroscopy-guided Procedures” (March 2018), arXiv:1803.08606, or any other suitable rendering process.

FIG. 6C illustrates an image representing a sectional view of volumetric imaging data representing the anatomical region. In this figure, the aorta 54 has been segmented. For example, the processing circuitry 24 is configured to segment the anatomical region, which in this embodiment comprises the aorta. The processing circuitry 24 is configured to use a segmentation method, such as a machine learning or convolution neural network (CNN) based method or another segmentation method, to segment the anatomical region. The processing circuitry 24 may be configured to automatically segment the anatomical region. The term “segment” may be interchangeably used with the term “mask.”

In some embodiments, a part of the segmentation of the anatomical region, the processing circuitry 24 is configured to set a value of each voxel adjacent to or surrounding the anatomical region to zero. The processing circuitry is configured to maintain of a value of each voxel, such as a measured Hounsfield unit value, that is part of the anatomical region.

In some embodiments, a part of the segmentation of the anatomical region, the processing circuitry is configured to set a value of each voxel adjacent to or surrounding the anatomical region to zero. The processing circuitry is configured to set a value of each voxel that is part of the anatomical region to a predetermined value, such as one.

The processing circuitry 24 is configured to project the segmented anatomical region onto an image plane to generate a 2D image representing the segmented anatomical region. In some embodiments, the processing circuitry 24 is configured to use the generated 2D image as the second map. In other embodiments, the processing circuitry 24 is configured to further process the generated 2D image to generate the second map.

The processing circuitry 24 is configured project the segmented anatomical region onto the image plane based on the plurality of registration parameters. The processing circuitry 24 is configured to project the segmented anatomical region using the DRR method described above. FIG. 6D illustrates a 2D image representing a segmented anatomical region. For example, in some embodiments where the processing circuitry is configured to set the value of each voxel adjacent to or surrounding the anatomical region to zero and the value of each voxel that is part of the anatomical region to the predetermined value, when the anatomical region is segmented, the generated 2D image representing the anatomical region may be a binary 2D image, such as the 2D image illustrated in FIG. 6D.

In some embodiment, the processing circuitry 24 is configured to apply a threshold to the 2D image representing the segmented anatomical region. For example, in embodiments where the processing circuitry is configured to maintain each value of each voxel that is part of the anatomical region, when the anatomical region is segmented, the processing circuitry is 24 is configured to apply the threshold to the generated 2D image representing the segmented anatomical region. This may generate a 2D binary image representing the segmented anatomical region, such as the 2D image illustrated in FIG. 6D. For example, the threshold may be a pixel value that divides the pixels of the 2D image representing the segmented anatomical region in at least a first part and a second part. The first part of pixels comprises one or more pixels having a pixel value that is higher than the threshold. The second part of pixels comprises one or more pixels having a pixels value that is lower than the threshold. For example, the threshold may be a pixel value that is larger than zero. For example, pixels that are part of the segmented anatomical region may be part of the first part of pixels. The processing circuitry 24 may be configured to assign a predetermined value, such as one, to each pixel of the first part of pixels. This may result in the generation of the 2D binary image.

In this embodiment, the processing circuitry 24 is configured to generate an outline 56 of the segmented anatomical region based on the generated 2D binary image representing the segmented anatomical region. The processing circuitry 24 is configured to project the outline 56 of the segmented anatomical region on the generated 2D image, as illustrated in FIG. 6D.

FIG. 6E illustrates the second 2D image shown in FIG. 3A including the outline 56 of the segmented anatomical region, e.g. the segmented aorta. The processing circuitry 24 is configured to project the outline of the segmented anatomical region onto the second 2D image. The processing circuitry 24 is further configured to project the outline 56 of the anatomical region onto the second 2D image based on the plurality of registration parameters. The outline 56 of the anatomical region may be projected on the second 2D image to illustrate the location of the anatomical region and/or an expected location of the medical device.

As described above, in some embodiments, the processing circuity 24 is configured to further process the generated 2D image representing the segmented anatomical region. For example, the processing circuitry 24 is configured to generate a distance map based on the generated 2D image representing the segmented anatomical region. In such embodiments, the processing circuitry is configured to use a distance from a centre of the segmented anatomical region to generate the distance map. FIG. 6F illustrates an exemplary distance map that has been generated based on the generated 2D image representing the segmented anatomical region, which is illustrated in FIG. 6D. In this embodiment, the distance map represents the second map.

Additionally or alternatively, a size or dimension of the segmented anatomical region may be varied, e.g. increased, as will be described below in more detail.

In other embodiments, the processing circuitry 24 is configured to use the 2D image representing the segmented anatomical region, e.g. as illustrated in FIG. 6D, as the second map. The calculation of the pixel value at each location where an X-ray is incident on the detector 52 is of similar nature as the distance transform mentioned above. This may allow for use of 2D image representing the segmented anatomical region as second map without the need for further processing.

In some embodiments, the processing circuitry 24 is configured to generate the second map based on one or more properties of the medical device. For example, the processing circuitry is configured to take into account the properties of the medical device, when determining the expected location of the medical device in the anatomical region. For example, the processing circuitry 24 is configured to encode one or more properties of the medical device in the second map. The properties of the medical device may comprise a minimum curvature of the medical device and/or another property of the medical device. The properties of the medical device may also be referred to as mechanical features.

As mentioned above, in some embodiments, the planning data is defined by a user. For example, the user may manually annotate the expected location and/or route of the medical device in the volumetric imaging data representing the anatomical region. The user may annotate the planning data in the volumetric imaging data by hand. The expected location and/or route of the medical device may be annotated as a spline or spline function in the volumetric imaging data representing the anatomical region. Alternatively, expected location and/or route may be annotated using a point annotation method or tool or another suitable annotation method or tool.

FIG. 7 illustrates an exemplary spline representing an expected location and/or route of the medical device in the volumetric imaging data representing the anatomical region. In the example illustrated in FIG. 7, the spline 55 is piecewise defined by three polynomials to interpolate between points P0 to P2. However, it will be appreciated that in other embodiments, the spline may be defined by more or less than three polynomials.

In such embodiments, the processing circuitry 24 is configured to define a 3D region in the volumetric imaging data representing the planning data. For example, the processing circuitry 24 is configured to define a 3D region, such as a cylindrical 3D region, along the spline. The processing circuitry 24 is configured to segment or extrude the 3D region from the volumetric imaging data. Any features described above in relation to the segmentation of the anatomical region may also apply to a segmentation of the 3D region.

The processing circuitry 24 is configured to project the segmented or extruded 3D region onto an image plane to generate the second map. In this embodiment, the segmented or extrudes 3D region replaces the segmented anatomical region mentioned above. As such, the processing circuitry 24 is configured to use and/or process the segmented or extruded 3D region in the same manner as the segmented anatomical region described above.

In use, there may be an intrinsic uncertainty in the alignment between the expected location and the likely location of the medical device in the anatomical region. This may be due to the medical device lying anywhere in the anatomical region and/or the anatomical region moving between the acquisition of the volumetric imaging data and the first 2D image, e.g. due to the patient's breathing, heart motion or the like. The generation of the second map described herein may allow for this intrinsic uncertainty in alignment. As mentioned above, the size or dimension of the segmented anatomical region or the segmented or extruded 3D region may be increased. This may provide an increased flexibility in the alignment between the expected location and the likely location of the medical device in the anatomical region. This will be further described below in relation to FIGS. 8A to 8E.

FIG. 8A illustrates a 2D image representing a simulated medical device 42a. The simulated medical device 42 is provided in the form of a guide wire.

FIG. 8B illustrates a 2D image representing a simulated anatomical region. The simulated anatomical region is provided in the form of a simulated aorta 54a.

FIG. 8C illustrates a 2D image representing a difference between the 2D image illustrated in FIG. 8A and the 2D image illustrated in FIG. 8B. The 2D image illustrated in FIG. 8C has been obtained by subtracting the 2D image illustrated in FIG. 8B from the 2D image illustrated in FIG. 8A. It can be seen from FIG. 8C that the guide wire and the aorta are not aligned, which may affect an accuracy of the 2D/3D registration. For example, a capture range of the image metrics may be narrow, which may provide the processing circuitry 24 with a decreased range of initial registration parameters for optimising or updating. This may result in inaccurate registration parameters for initialising the 2D/3D registration.

FIG. 8D illustrates a 2D image representing a simulated anatomical region with an increased size or dimension of the simulated anatomical region. As described above, in some embodiments, the processing circuitry 24 is configured to increase a size or dimension of the segmented anatomical region or the segmented or extruded 3D region. The size or dimension of the segmented anatomical region or the segmented or extruded 3D region may be increased, e.g. prior to projecting the segmented anatomical region onto the image plane. Alternatively, the size or dimension of the segmented anatomical region or the segmented or extruded 3D region may be increased subsequently to projecting the segmented anatomical region onto the image plane. For example, the processing circuitry 24 may be configured to further process the generated 2D image representing segmented anatomical region or the segmented or extruded 3D region by increasing the size or dimension of the segmented anatomical region or the segmented or extruded 3D region. In this embodiment, the processing circuitry is configured to dilate anatomical region, e.g. the simulated aorta 54a. The increase of the size or dimension of the segmented anatomical region may also be referred to as dilating the segmented anatomical region.

FIG. 8E illustrates a 2D image representing a difference between the 2D image illustrated in FIG. 8A and the 2D image illustrated in FIG. 8D. The 2D image illustrated in FIG. 8E has been obtained by subtracting the 2D image illustrated in FIG. 8D from the 2D image illustrated in FIG. 8A. It can be seen from FIG. 8E that the dilation of the simulated aorta 54a increases a range of the aorta. For example, by increasing the size or dimension of the anatomical region, a capture range of the image metrics may be increased, e.g. compared to that in the example shown FIG. 8C. This may provide the processing circuitry 24 with an increased range of initial registration parameters for optimising or updating. This in turn may result in a more robust and/or improved Initialisation of the 2D/3D registration.

In some embodiments, the processing circuitry 24 is configured to project the planning data onto one or more first 2D images and/or the second 2D image based on the plurality of updated registration parameters. The one or more first 2D images may comprise the first 2D image and/or one or more subsequent first 2D image representing the medical device in the anatomical region. The first 2D image and/or the subsequent first 2D images may be part of the 2D image sequence, e.g. the live 2D image sequence.

FIG. 9A illustrates a 2D image representing an anatomical region of a patient or other subject. The 2D image illustrated in FIG. 9A is a digitally reconstructed radiograph that has been generated from the volumetric image data representing the anatomical region. In FIG. 9A, the planning data comprises an access route 58 and a location of an anatomical structure 60, which in this embodiment comprises an aortic valve.

FIG. 9B illustrates a 2D image representing the anatomical region. The 2D image shown in FIG. 9B has been acquired using the fluoroscopy method described above. In FIG. 9B, the planning data, e.g. the access route 58 and the location of the anatomical structure 60, has been projected onto the first 2D image. In this example, the medical device 42 can be seen as being located in the access route 58 and extending into the location of the anatomical structure 60. This may aid a user with the medical procedure to be carried out. It will be appreciated that the processing circuitry 24 may be configured to additionally or alternatively project the planning data on one or more subsequent first 2D images of the 2D image sequence.

Certain embodiments provide a medical image processing apparatus comprising processing circuitry configured to receive a medical image volume (CT), a 2D medical image sequence (X-ray etc), and details of the intervention/procedure type (e.g. TAVI), obtain tools/instruments in the 2D image(s), using pre-operative planning information, generate 2D projected “tool probability maps” from the 3D CT volume, optimise 2D/3D registration parameters to update the probability map to best align with tools in the 2D image, and use optimal registration parameters to project planning information onto the 2D image(s).

The interventional objects in the 2D image may be detected automatically. The tools may be detected by classical filtering operations (e.g. smoothing and vesselness filter). The tools may be detected by machine learning (e.g. CNN).

The interventional objects in the 2D image may be annotated by hand (point annotation, spline etc).

The interventional procedure may obtain access via the aorta (e.g. TAVI).

The aorta may be segmented automatically.

The aorta may be projected to 2D space without modification.

The projected image may be further processed (some combination of binarized, dilated, distance map) to produce a 2D probability map.

The probability map encodes mechanical features of the probe used (minimum curvature path, racing line etc).

The aorta may be dilated prior to projecting the aorta to 2D space.

The intervention access route may be manually annotated. The intervention access route may be automatically determined, based on a standardised plan for the intervention.

The interventional procedure may obtain access via another route (where either automatic segmentation of the anatomy corresponding to the access pass is obtained or the route is manually obtained).

The subsequent “live” fluoroscopy registration may be constrained to parameters close to the initialisation.

It will be appreciated that the terms “volumetric image data” and “volumetric imaging data” may be interchangeably used. The volumetric image data may also be referred to as a 3D CT volume.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical image processing apparatus comprising processing circuitry configured to:
generate a first map representing a likely location of a medical device in an anatomical region of a patient or other subject, the first map being generated based on a two-dimensional (2D) image that has been acquired by a medical imaging apparatus, the 2D image representing the medical device in the anatomical region;
generate a second map representing an expected location of the medical device in the anatomical region, the second map being generated based on volumetric imaging data representing the anatomical region and a plurality of registration parameters, wherein the plurality of registration parameters determines a pose of the medical imaging apparatus;
compare the first and second maps with each other; and
update the plurality of registration parameters based on the comparison between the first and second maps.

2. The apparatus of claim 1, wherein the processing circuitry is configured to initialise a registration of at least one of: the medical imaging apparatus and/or a 2D image sequence with the volumetric imaging data based on a plurality of updated registration parameters, the 2D image sequence comprising at least the 2D image and/or one or more subsequent 2D images representing the medical device in the anatomical region.

3. The apparatus of claim 1, wherein the expected location of the medical device in the anatomical region is part of planning data and the processing circuitry is configured to generate the second map based on the planning data.

4. The apparatus of claim 3, wherein the processing circuitry is configured to project the planning data onto one or more 2D images based on a plurality of updated registration parameters, the one or more 2D images comprising at least the 2D image and/or one or more subsequent 2D images representing the medical device in the anatomical region.

5. The apparatus of claim 3, wherein the processing circuitry is configured to determine the planning data based on a medical procedure to be carried out.

6. The apparatus of claim 3, wherein the planning data is defined by a user.

7. The apparatus of claim 3, wherein the planning data comprises at least one of: a route of the medical device in the anatomical region and a location of an anatomical structure.

8. The apparatus of claim 5, wherein the medical procedure to be carried out comprises a transcatheter aortic valve implantation procedure and the anatomical region comprises an aorta of a patient or other subject.

9. The apparatus of claim 1, wherein the processing circuitry is configured to perform one or more iterations of at least one of: generating the first map, generating the second map, comparing the first and second maps with each other and updating the plurality of registration parameters based on the comparison between the first and second maps.

10. The apparatus of claim 9, wherein in a first iteration, the plurality of registration parameters comprises a plurality of initial registration parameters, and in each subsequent iteration, the plurality of registration parameters comprises a plurality of updated registration parameters, the plurality of updated registration parameters comprising at least one registration parameter that has been updated relative to a corresponding previous registration parameter.

11. The apparatus of claim 9, wherein in each subsequent iteration, the processing circuitry is configured to generate the second map based on a plurality of updated registration parameters.

12. The apparatus of claim 1, wherein the processing circuitry is configured to detect the medical device in the 2D image.

13. The apparatus of claim 12, wherein the processing circuitry is configured to use a filtering method or a machine learning method to detect the medical device in the 2D image.

14. The apparatus of claim 1, wherein the processing circuitry is configured to segment the anatomical region in the volumetric imaging data.

15. The apparatus of claim 14, wherein the processing circuitry is configured to project the segmented anatomical region onto an image plane to generate an image representing the segmented anatomical region, the segmented anatomical region being projected onto the image plane based on the plurality of registration parameters.

16. The apparatus of claim 15, wherein the processing circuitry is configured to at least one of:
generate a binary image representing the segmented anatomical region based on the generated image;
increase a size or dimension of the segmented anatomical region; and
generate a distance map based on the generated image.

17. The apparatus of claim 1, wherein the processing circuitry is configured to generate the second map based on one or more properties of the medical device.

18. The apparatus of claim 1, wherein the 2D image comprises a fluoroscopic image, a range of a field of view of the 2D image being reduced to focus on or zoom in the anatomical region.

19. The apparatus of claim 1, wherein the anatomical region comprises an artery, vein or an organ of the patient or other subject, the organ comprising a cylindrical or tubular shape.

20. A medical image processing method comprising:
generating a first map representing a likely location of a medical device in an anatomical region of a patient or other subject, the first map being generated based on a two-dimensional (2D) image that has been acquired by a medical imaging apparatus, the 2D image representing the medical device in the anatomical region;
generating a second map representing an expected location of the medical device in the anatomical region, the second map being generated based on volumetric imaging data representing the anatomical region and a plurality of registration parameters, wherein the plurality of registration parameters determines a pose of the medical imaging apparatus;
comparing the first and second maps with each other; and
updating the plurality of registration parameters based on the comparison between the first and second maps.
